(19) United States
(12) Patent Application Publication
(10) Pub. No.: US 2010/0125282 A1
(43) Pub. Date: May 20, 2010

(54) Robotically Steered RF Catheter

(75) Inventors: James Machek, Santa Rosa, CA (US); Michael Francis, Loughborough (GB)

Correspondence Address: MEDTRONIC VASCULAR, INC.;IP LEGAL DEPARTMENT, 3576 UNOCAL PLACE, SANTA ROSA, CA, 95403, US

(73) Assignee: MEDTRONIC VASCULAR, INC., Santa Rosa, CA (US)

(21) Appl. No.: 12/271,493

(22) Filed: Nov. 14, 2008

(51) Int. Cl. A61B 19/00; A61M 5/00

(52) U.S. Cl. 606/130; 606/108

(57) ABSTRACT

A distal end of a stent graft fenestration or other special purpose catheter is guided to a location in a patient's vasculature based on input from a surgeon using a pre-operatively generated image of the patient's vasculature—this can be in proximity to an already implanted device. The movement of the catheter is controlled by a robotic surgical system based upon the input from the surgeon. The distal end of the catheter, at the location, is synchronized to the patient's cardiac cycle so the robotic surgical system maintains the distal end of the catheter at the location throughout a cardiac cycle of the patient. The surgeon is not required to precisely manipulate the catheter to maintain the distal end in the proper location throughout the cardiac cycle. The surgeon can focus on performing the desired procedure such as fenestrating the stent graft, endostapling or endosuturing, for example.

### BACKGROUND OF THE INVENTION

1. Field of the Invention

The present invention relates generally to use of stent-grafts, and more particularly to devices used for fenestration of a stent-graft in situ.

2. Description of Related Art

A conventional main (vessel) stent-graft typically includes a radially expandable reinforcement structure, formed from a plurality of annular stent rings, and a tubular shaped graft material, sometimes called graft cloth, defining a lumen, to which the stent rings are coupled. The stent rings includes straight portions that are referred to as struts. Main stent-grafts are well known for use in tubular shaped human vessels.

To illustrate, endovascular aneurysmal exclusion is a method of using a main stent-graft to exclude pressurized fluid flow from the interior of an aneurysm. This reduces the risk of rupture of the aneurysm and the associated risk of mortality.

Main stent-grafts with custom side openings are sometimes fabricated to accommodate particular vessel geometries of each individual patient. Specifically, as the location of branch vessels emanating from a main vessel, e.g., having the aneurysm, varies from patient to patient, main stent-grafts to treat such configurations are fabricated with side openings customized to match the position of the branch vessels of the particular patient. However, custom fabrication of main stent-grafts is relatively expensive and time consuming.

To avoid custom fabrication of main stent-grafts, side openings in the main stent-graft may be formed in situ. Illustratively, the main stent-graft is placed in the main vessel, e.g., the aorta, to exclude an aneurysm. Fenestrations may be made in situ to correspond to positions of the adjacent branch vessels. See U.S. Pat. No. 5,617,878 of Taheri.

The graft material of the main stent-graft is pierced using a fenestrating catheter with a needle at the ostium of a branch vessel, e.g., the renal artery, emanating from the main vessel. A fenestration is typically initiated with a small needle perforation of the graft material. The perforation must be enlarged with a conical dilator. However considerable force is required to push the dilator through the graft material and the application and use of such considerable force in a sideways direction at the end of a catheter is difficult to control and can cause the graft material to deflect and move and/or suddenly tear causing the unexpected dilator motion which can damage or pierce the vessel wall opposite the dilator, which is not desirable.

Once the dilator opening has been made, an expandable balloon is inserted in the opening in the graft material of the main stent-graft and the balloon inflated to tear or cut the graft material further.

If the fenestration catheter utilizes a small RF electrode, rather that a dilator to create the initial opening for the balloon, the initial application of force is reduced, however in both instances the use of the balloon cause tearing and fraying of the graft material. The use of a balloon to enlarge a graft opening is difficult to control and thus leads to unpredictability in the tear of the graft material or other complications. Further, the branch stent-graft tends to propagate the rent (a split or tear) in the graft material over time. Also, the edge of the rent fractured the branch stent-graft depending upon the particular application. Finally, the edge of the rent was a fray of loose fibers of the graft material, which tended to unwind over time.

### SUMMARY OF THE INVENTION

A method for in-situ fenestration of a stent graft overcomes the prior problems. In one aspect, a distal end of a (fenestration initiating) catheter is guided to a location in a patient's vasculature based on input from a surgeon using a pre-operatively generated image of the patient's vasculature. The movement of the catheter is controlled by a robotic surgical system based upon the input from the surgeon.

The position of the distal end of the catheter, at the location, is synchronized to the patient's cardiac cycle so the robotic surgical system maintains the position of the distal end of the catheter at a selected dimension from or relative to the location throughout a cardiac cycle of the patient. Thus, the surgeon is not required to precisely manipulate the catheter to maintain the distal end in the desired location throughout the cardiac cycle. The surgeon can focus on performing the desired procedure such as fenestrating the stent graft, endostapling, or endosuturing, for example.

The method also includes generating the pre-operative image, prior to the catheter being guided, for a particular point is the cardiac cycle. In addition, the method positions, prior to the catheter being guided, the distal end of the catheter at a known location in the patent's vasculature. The known location is different from the location. Next, the method, synchronizes, prior to the catheter being guided, an in-vivo image of the patient's vasculature to the pre-operatively generated image. The in-vivo image is of the known location and is taken at a point in the cardiac cycle corresponding to a point in the cardiac cycle for which the pre-operatively generated image was generated.

In another example, a method generates a pre-operative image of a patient's vasculature for a particular point in a cardiac cycle of the patient. A stent graft is placed in the patient's vasculature using the known procedure for such placement.

Next, a distal end of a robotically controlled catheter in a robotic surgical system is placed at a known location in the patient's vasculature. An image of the patient's vasculature is taken at the known location and at the particular point in the cardiac cycle.

The pre-operative image is synchronized to this image so as to synchronize the pre-operative image to the cardiac cycle. Following the image synchronization, the distal end of the robotically controlled catheter is guided to a fenestration location by a surgeon using the pre-operative image of the patient's vasculature and using the robotic surgical system. The fenestration location is where the stent graft requires fenestration.

The distal end of the robotically controlled catheter, at the fenestration location, is synchronized to the patient's cardiac cycle. Following the synchronizing, the robotic surgical system maintains the position of the distal end of the catheter a surgeon selected distance from the fenestration location throughout the cardiac cycle. The stent graft is fenestrated, at the fenestration location, using an ablating device at the distal end of the catheter by the surgeon instructing the robotic device to reduce the selected distance between the distal end of the catheter and the fenestration location until the ablation device at the distal end of the catheter comes in contact with the fenestration location without the surgeon manipulating the robotically controlled catheter to account for fenestration location movement due to the cardiac cycle.

In another aspect, a robotic surgical system is configured to perform a method comprising: guiding a distal end of a catheter to a location in a patient's vasculature based on input from a surgeon using a pre-operatively generated image of the patient's vasculature, wherein the catheter is controlled by a robotic surgical system based on the input from the surgeon; and synchronizing the distal end of the catheter, at the location to the patient's cardiac cycle so the robotic surgical system maintains the catheter at the location throughout a cardiac cycle of the patient.

### BRIEF DESCRIPTION OF THE DRAWINGS

FIG. 1 is a block diagram of a robotic surgical system.

FIG. 2 is a process flow diagram for a method for using a pre-operative image of a patient's vasculature and the robotic surgical system of FIG. 1 to perform an in-situ procedure.

FIG. 3 is a process flow diagram for a method for using a pre-operative image of a patient's vasculature and the robotic surgical system of FIG. 1 to perform in situ fenestration of a stent graph.

### DETAILED DESCRIPTION

In one implementation according to the present invention, when a main stent-graft (representative of a device previously implanted in the patient's vasculature) is placed in a main vessel of a patient and a branch vessel is blocked by the main stent-graft, a robotically controlled (fenestration) catheter including a mechanically actuated radio-frequency (RF) ablation device is steered by a surgeon by using a display 141 of a pre-operative image 131 of the patient's vasculature and a manipulator 142 of robotic surgical system 100. The RF ablation device is positioned adjacent to an ostium of the branch vessel to be perfused and is synchronized with the patient's cardiac cycle. As used herein, a cardiac cycle is the flow of blood from one heartbeat to the next heartbeat.

Robotic surgical system 100 then according to its automated functional instructions maintains a point on the RF ablation device (such as its distal end) in the proper position (a surgeon selected distance) with respect to the ostium of the branch vessel during the cardiac cycle. Thus, the surgeon is not required to precisely manipulate the RF ablation device to maintain the device in the proper location.

The RF ablation device is used to cut out a portion of the graft cloth of the main-stent graft adjacent to the ostium so that the branch vessel is perfused. This allows the fenestration to be made in situ in a matter of minutes, at most, following placement of the main stent-graft. RF ablation devices suitable for use in the method include those described in U.S. patent application Ser. No. 12/106,677, entitled “A Family of Electrodes for Use in Performing in Situ Fenestration Using a Plasma RF Catheter,” of Walter Bruszewski et al., filed on Apr. 21, 2008, which is incorporated herein by reference in its entirety.

While fenestrating a stent graft is used as an example, of using a pre-operative image of a patient's vasculature and a robotic surgical system to guide a device to a particular location and synchronize maintaining the position of that device's distal end (or other element) at a particular position (selected dimension from a treatment or treatment target location) throughout the cardiac cycle of the patient, in other applications, a similar procedure can be used for endostapling and/or endosuturing or other procedures performed within the patient's vasculature using a robotically guided catheter.

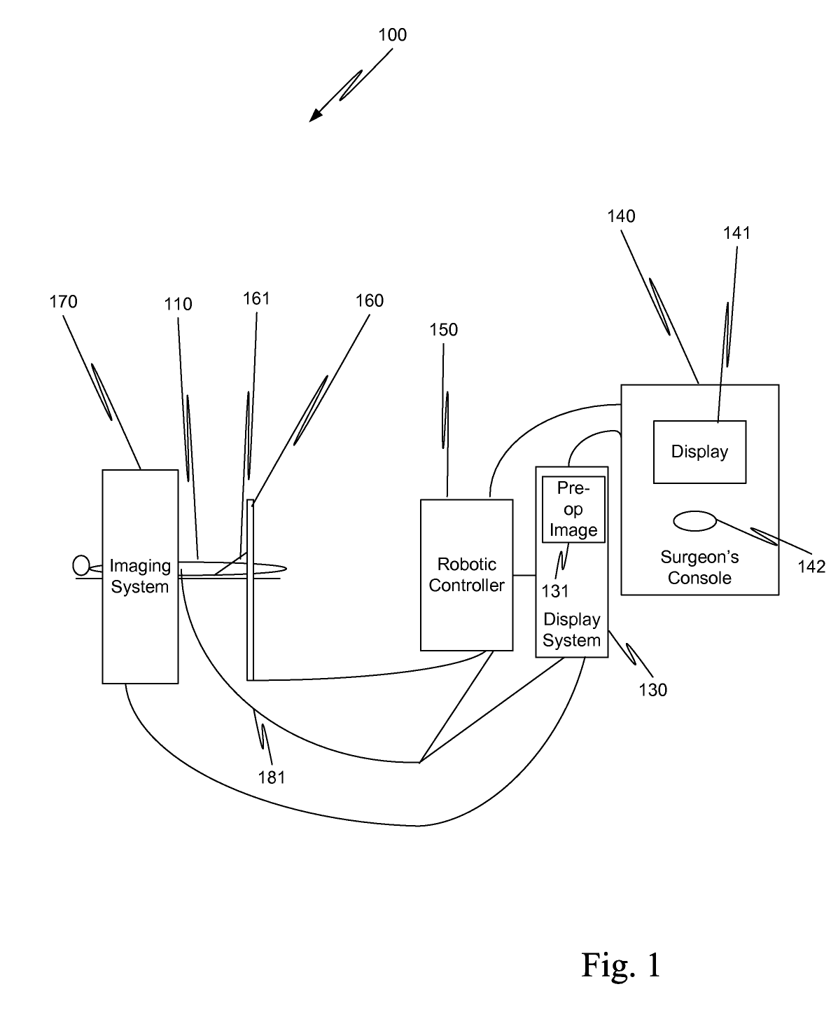

FIG. 1 is a block diagram of a robotic surgical system 100. Robotic surgical system 100 includes a surgeon's console 140, a robotic platform 160 with a robotic manipulator arm 161, a robotic controller 150 and a display system 130. Surgeon's console 140 includes a display 141 and a manipulator 142. A surgeon operates manipulator 142. In response to the signals from manipulator 142, robotic controller 150 sends signals to robotic manipulator arm 161 that in turn appropriately moves a catheter that has been inserted in patient 110 via the femoral artery, for example.

Robotic surgical systems for controlling catheters are known to individuals knowledgeable in the field and so system 100 is not described in further detail. Also, the supplier of the robotic surgical system typically supplies a catheter that can be used with the system One robotic surgical system with a catheter suitable for use with the examples described herein is available from Hansen Medical, 380 North Bernardo Avenue, Mountain View, Calif., USA.

In this example, a display system 130 provides information to display 141. Sensors are attached to patient 110 to provide electrocardiogram signals 181 to either or both of robotic controller 150 and display system 130. Those knowledgeable in the field understand that robotic controller 150 and display system 130 may be a combination of hardware, computer programs, one or more computer processors, and other sensors and maybe separate units or combined in a single unit.

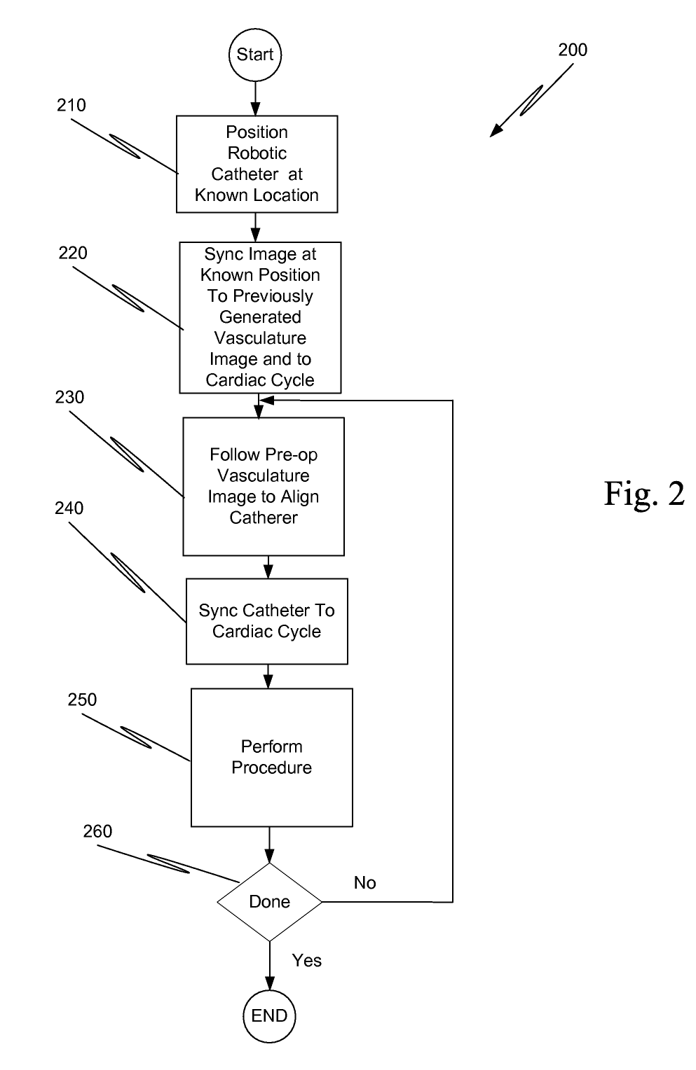

In one example, in process 210 of a method 200 (FIG. 2) a catheter (not shown) having a device attached at a distal end of the catheter is entered into the vasculature system of patient 110 and robotically positioned, using robotic surgical system 100, at a known location. As used herein, the distal end of the catheter is the end furthest removed from robotic manipulator arm 161.

Typically, a radio-opaque marker or an antenna is placed at the distal end of the catheter and imaging system 170 is used to determine when the distal end of the catheter is positioned at the known location. An image of the known location in the vasculature of patient 110 and at a known point in the cardiac cycle of patient 110 is taken and is referred to as an in-vivo image. Cardiac gating, using electrocardiogram signal 181 from patient 110, to capture or generate an image at a particular point in the cardiac cycle is known to those knowledgeable in the field.

As described above, display system 130 includes a pre-operative image 131 of the vasculature of patient 110 that was taken at known point in the cardiac cycle and that includes the known location. Thus, in process 220, the in-vivo image is synchronized with pre-operative image 131. Thus, pre-operative image 131 is synchronized to the cardiac cycle of the patient. Pre-operative image 131 to be used by the surgeon, in a manner similar to the way a roadmap is used, to guide the distal end of the catheter to a location where the procedure is to be performed.

Thus, in follow pre-operative vasculature image to align catheter process 230, the surgeon uses manipulator 142 and the pre-operative vasculature image on display 141 to align the device on the distal end of the catheter with the location where the surgical procedure is to be performed. With the device in the approximate location, the location of the device is synchronized to the patient's cardiac cycle in process 240. This synchronization permits robotic controller 150 to keep the distal end in the desired location with respect to a point on the body structure (or fenestration location) as selected by the surgeon on the in-vivo image throughout the cardiac cycle.

In perform procedure 250, the surgeon uses the device to perform the desired surgical procedure without having to worry about precisely manipulating the device to maintain the position the device in a particular location throughout the cardiac cycle. Robotic controller 150 automatically and according to its specific functional instructions performs the necessary manipulations to maintain the specified portion of the catheter device a selected or minimum closest approach distance to a treatment target (or fenestration) location that correspond with the cardiac cycle.

If the surgeon has completed all the procedures needed using the device, the processing is done and so done check operation 260 passes to end where the robotic surgical procedure is completed in the normal way. Conversely, if other procedures are needed, e.g., additional fenestrations, endosutures, or endostaples, processes 230 to 250 are repeated as needed.

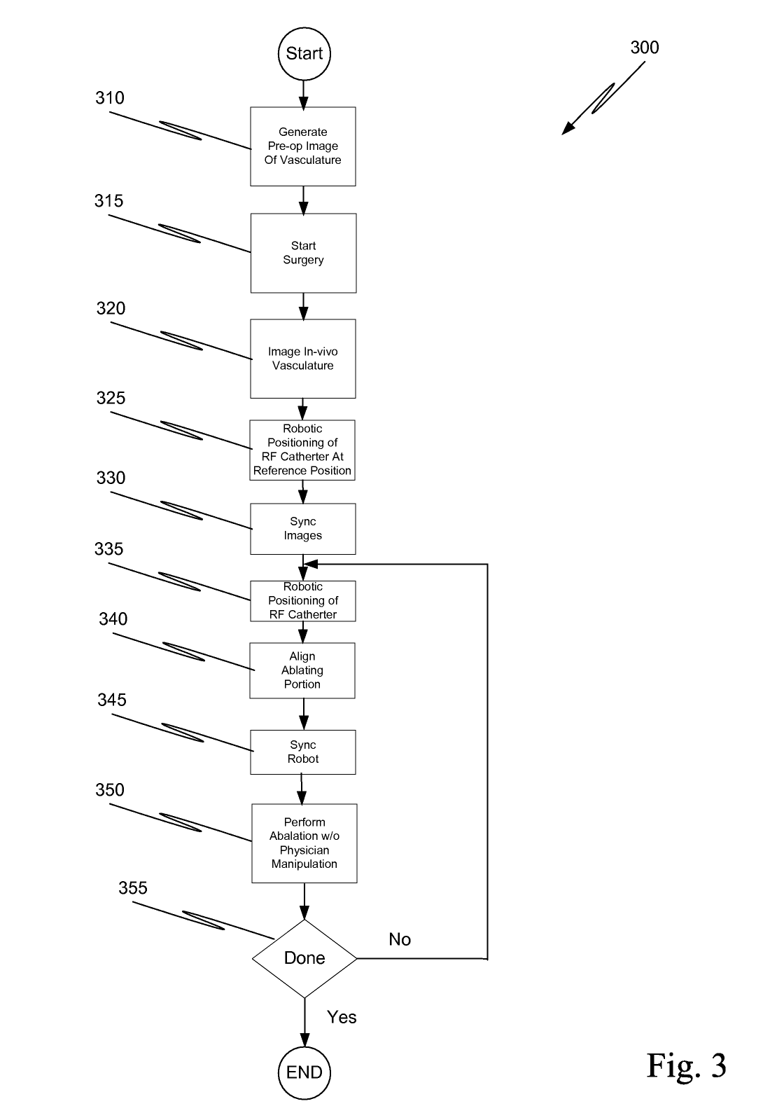

FIG. 3 is a more detailed process diagram for a method 300 of using robotic surgical system 100 to perform an in-situ fenestration of a stent graft. Prior to the surgery to insert the stent graft, generate pre-operative image of patient's vasculature process 310 is performed. In process 310, for example, a number of computer tomography (CT) scan slices are taken of the patient's vasculature. These slices are used to create a three-dimensional endovascular image of the patient's vasculature.

Specifically, an endovascular image can be formed using retrospective electrocardiogram gating, sometimes called cardiac gating. In this process, each portion of the heart and adjacent vasculature is imaged more than once by the CT system while an electrocardiogram trace is recorded. The electrocardiogram trace is then used to correlate the CT data with the corresponding phases of the cardiac cycle. Once this correlation is complete, all data that were recorded while the heart was in motion, for example, are ignored and the three-dimensional endovascular image is made from the remaining data that were acquired while the heart was at rest.

In another example, image acquisition is triggered by a start pulse derived from an electrocardiogram taken from the patient while imaging. For imaging during diastole, the electrocardiogram trace is fed into a circuit which produces a trigger signal during diastole that in turn is used as a start signal for data acquisition by the imaging system. The imaging system then automatically acquires data for a time series of images of the patient's vasculature at a particular point in the cardiac cycle. Cardiac gating methods typically yield suboptimal results whenever the patient has an irregular heartbeat.

The particular technique used to obtain the data for generating the three-dimensional pre-operative image of the patient's vasculature at a point in the cardiac cycle is not critical so long as the data can be used to form a three-dimensional endovascular image that can be implemented in the robotic surgical system and synchronized as described more completely below. One company that provides a system for generating a volumetric endovascular image suitable for use in this invention is M2S of 12 Commerce Avenue, West Lebanon, N.H., USA.

At some time following completion of generate pre-operative image of vasculature process 310, start surgery process 315 is performed. In one example, in process 315 a stent graft is placed in an abdominal aortic aneurysm and one or more fenestrations of the stent graft are required to profuse secondary arteries.

Robotic surgical system 100 is used to insert a mechanically actuated radio-frequency (RF) ablation device, sometimes referred to as the ablating device, mounted on a distal end of a catheter into patient 110. As indicated above, the distal end is the end furthest removed from robotic manipulator arm 161. The mechanically actuated radio-frequency ablation device has an antenna that is used to determine the location of the device within the patient's vasculature.

Display system 130 uses the information stored as pre-operative image 131 to present a three-dimensional image in display 141 on surgeon's console 140. In image in-vivo vasculature process 320, the surgeon utilizes elements on surgeon's console 140 to cause imaging system 170 to generate an image or images of the patient's vasculature. As described above, the electrocardiogram signals on the line carrying the electrocardiogram signals 181 can be used to gate the production of the image by imaging system 170 so that the image is correlated with a particular point in the cardiac cycle. Imaging system 170 can be, for example, one of a CT system or a fluoroscope.

Using the in-vivo image or images and the tracking of the distal end of the catheter, the surgeon uses manipulator 142 to cause robotic controller 150 to position the distal end of the catheter at a known reference location in the patient's vasculature in process 325. With the distal end of the catheter positioned at the known reference location, this identifies the location with respect to the image of the in-vivo vasculature.

Thus, display system 130 synchronizes the in-vivo image with the pre-operative image. For example, if the pre-operative image was generated using imaging system 170 shortly before the surgery, the single reference point is sufficient to overlay the in-vivo image on the pre-operative image with respect to the known location at the correct point in the cardiac cycle. With this synchronization, the pre-operative image stays fixed with the patient's vasculature throughout the cardiac cycle. This in turn permits the surgeon to use the display of the pre-operative image in guiding the distal end of the catheter using robotic system 100 in process 335.

However, if the pre-operative image was generated sometime before the surgery and/or on a different imaging system, a set of reference points, e.g., three reference points, are used in synchronize image process 330 to align the pre-operative image with the in-vivo images and to thereby synchronize the pre-operative image to the patient's cardiac cycle. This in turn permits the surgeon to use the display of the pre-operative image in guiding the distal end of the catheter using robotic system 100 in process 335.

When the surgeon has caused the distal end of the catheter to be moved to the desired position using system 100, the surgeon uses manipulator 142 to position the ablating device in alignment with the ostium of the secondary vessel to be perfused in align ablating portion process 340. In sync robot process 345, while maintaining the ablating device in the proper position, the surgeon uses elements on surgeon's console 140 to cause robotic controller 150 to synchronization the position of the ablating device at the appropriate point in the cardiac cycle so that that robotic controller 150 can then keep the ablating device aligned with the ostium of the secondary vessel to be perfused throughout the cardiac cycle. Consequently, the surgeon can focus on the fenestration and does not have to be concerned with manipulations to maintain proper positioning of the ablating device throughout the cardiac cycle.

In process 350, the surgeon actuates the ablating device to perform fenestration of the stent graft without having to manipulate the ablating device to maintain the device in the proper location. The ablating device takes on the order of ten or so nanoseconds to perform the fenestration.

If the surgeon has completed all the needed fenestrations, the procedures are complete and so done check operation 355 passes to end where the robotic surgical procedure is completed in the normal way. Conversely, if other fenestrations are needed, processes 335 to 350 are repeated as needed.

The examples presented herein are illustrative only and are not intended to be limiting. In view of this disclosure, the principles can be used in a wide variety of robotic surgeries.

We claim:

1. A method comprising:
guiding a distal end of a catheter to a location in a patient's vasculature based on input from a surgeon using a pre-operatively generated image of the patient's vasculature, wherein said catheter is controlled by a robotic surgical system;
synchronizing said distal end of said catheter, at said location to the patient's cardiac cycle so said robotic surgical system maintains said catheter at said location throughout a cardiac cycle of the patient.

2. The method of claim 1 further comprising:
generating said pre-operatively generated image, prior to said guiding, for a particular point in said cardiac cycle.

3. The method claim 1 further comprising:
positioning, prior to said guiding, said catheter at a known location in said patient's vasculature, wherein said known location is different from said location.

4. The method of claim 3 further comprising:
synchronizing, prior to said guiding, an in-vivo image of said patient's vasculature to said pre-operatively generated image, wherein said in-vivo image is of said known location and is taken at a point in said cardiac cycle corresponding to a point in said cardiac cycle for which said pre-operatively generated image was generated.

5. The method of claim 1 further comprising:
performing, following said synchronizing said distal end of said catheter, a procedure at said location.

6. The method of claim 5 wherein said procedure comprises fenestrating a stent graft.

7. The method of claim 5 wherein said procedure comprises endostapling.

8. The method of claim 5 wherein said procedure comprises endosuturing.

9. A method comprising:
generating a pre-operative image of a patient's vasculature for a particular point in a cardiac cycle of the patient;
placing a stent graft in said patient's vasculature;
positioning a distal end of a robotically controlled catheter in a robotic surgical system at a known location in said patient's vasculature;
taking an image of said patient's vasculature at said known location and at the particular point in the cardiac cycle;
synchronizing said pre-operative image to said image so as to synchronize said pre-operative image to said cardiac cycle;
guiding said distal end of said robotically controlled catheter to a fenestration location using said pre-operative image of the patient's vasculature, wherein said fenestration location requires fenestration of said stent graft;
synchronizing said distal end of said robotically controlled catheter, at said fenestration location, to the patient's cardiac cycle wherein following said synchronizing, said robotic surgical system maintains said distal end of said robotically controlled catheter at said fenestration location throughout the cardiac cycle;
fenestrating said stent graft, at said fenestration location, using an ablating device at said distal end without a surgeon manipulating said robotically controlled catheter to maintain said ablating device at said fenestration location.

10. A robotic surgical system configured to perform a method comprising:
guiding a distal end of a catheter to a location in a patient's vasculature based on input from a surgeon using a pre-operatively generated image of the patient's vasculature, wherein said catheter is controlled by the robotic surgical system;
synchronizing said distal end of said catheter, at said location to the patient's cardiac cycle so said robotic surgical system maintains said catheter at said location throughout a cardiac cycle of the patient.

11. The robotic surgical system of claim 10, wherein said method further comprises:
positioning, prior to said guiding, said catheter at a known location in said patient's vasculature, wherein said known location is different from said location.

12. The robotic surgical system of claim 11, wherein said method further comprises:
synchronizing, prior to said guiding, an in-vivo image of said patient's vasculature to said pre-operatively generated image, wherein said in-vivo image is of said known location and is taken at a point in said cardiac cycle corresponding to a point in said cardiac cycle for which said pre-operatively generated image was generated.

13. The robotic surgical system of claim 10, wherein said method further comprises:
performing, following said synchronizing said distal end of said catheter, a procedure at said location.

14. The robotic surgical system of claim 13, wherein said procedure comprises fenestrating a stent graft.
